(19) United States
(12) Patent Application Publication
(10) Pub. No.: US 2020/0214649 A1
(43) Pub. Date: Jul. 9, 2020

(54) DETECTION OF COVID-19

(71) Applicant: THOMAS PAUL COGLEY, PINELLAS PARK, FL (US)

(72) Inventor: THOMAS PAUL COGLEY, PINELLAS PARK, FL (US)

(21) Appl. No.: 16/824,178

(22) Filed: Mar. 19, 2020

Related U.S. Application Data
(63) Continuation-in-part of application No. 16/168,990, filed on Oct. 24, 2018.

(51) Int. Cl. A61B 5/00; A61B 6/08; A61B 5/05; A61B 6/00; G16H 50/20; G16H 30/20

(52) U.S. Cl. CPC A61B 5/7445; A61B 6/08; A61B 5/05; G16H 30/20; A61B 5/746; A61B 5/7415; G16H 50/20; A61B 6/462

(57) ABSTRACT

The steps of the method are: providing a generator adapted to produce specific wavelengths to maximize light emissions; providing a scintillator in operative proximity to the generator, the scintillator having an associated scintillator screen of a specific phosphor or other excitive type sensitive to various wavelengths, the scintillator being sensitive to a wavelength that maximizes light emissions; positioning a patient to be diagnosed between the generator and the scintillator; emitting a specific wavelength from the generator to and through the patient onto the scintillator screen whereby the associated scintillator screen will light up and sparkle to produce a light image of the patient; providing a camera in operative proximity to the scintillator screen to record the light image produced on the scintillator screen; providing a computer with a computer screen and software; capturing and analyzing the recorded light images from the camera; and obtaining a diagnosis for treatment of the patient.

### RELATED APPLICATIONS

This application is a continuation-in-part of U.S. patent application Ser. No. 16/168,990 filed Oct. 24, 2018 and is based upon Provisional Application No. 62/982,404 filed Feb. 27, 2020, which are incorporated herein by reference and the priority of which are claimed.

### BACKGROUND OF THE INVENTION

### Field of the Invention

The present invention relates to the detection of inflammation associated with COVID-19 using a light wave. Included is methodology to alter and manipulate the light wave to produce a detector that is unequaled by any other device. It will provide the optimal detection method for the present emergency situation.

### Description of the Prior Art

The use of imaging systems and methods of known designs and configurations are known in the prior art. More specifically, imaging systems and methods of known designs and configurations previously devised and utilized for the purpose of checking for disease in a body are known to consist basically of familiar, expected, and obvious structural configurations and steps, notwithstanding the myriad of designs encompassed by the crowded prior art which has been developed for the fulfillment of countless objectives and requirements.

While these devices and methods fulfill their respective, particular objectives and requirements, they do not describe a detection of COVID-19 method that allows medical professionals to easily visualize aspects presently hidden to them on current imaging systems.

In this respect, the detection of CovID-19 method, according to the present invention substantially departs from the conventional concepts and designs of the prior art, and in doing so provides a method primarily developed for the purpose of allowing medical professionals to easily visualize aspects presently hidden to them on current imaging systems. Therefore, it can be appreciated that there exists a continuing need for a new and improved detection of COVID-19 method, which can be used for allowing medical professionals to easily visualize aspects presently hidden to them on current imaging systems. In this regard, the present invention substantially fulfills this need.

### SUMMARY OF THE INVENTION

In view of the disadvantages inherent in the known types of imaging systems and methods of known designs and configurations now present in the prior art, the present invention provides an improved detection of COVID-19 method. As such, the general purpose of the present invention, which will be described subsequently in greater detail, is to provide a new and improved detection of COVID-19 method which has all the advantages of the prior art and none of the disadvantages.

To attain this, from a broad perspective, the method of the present invention essentially comprises the steps of providing a generator adapted to produce specific wavelengths to maximize light emissions; providing a scintillator in operative proximity to the generator, the scintillator having an associated scintillator screen of a specific phosphor or other excitive type sensitive to various wavelengths, the scintillator being sensitive to a wavelength that maximizes light emissions; positioning a patient to be diagnosed between the generator and the scintillator; emitting a specific wavelength from the generator to and through the patient onto the scintillator screen whereby the associated scintillator screen will light up and sparkle to produce a light image of the patient; providing a camera in operative proximity to the scintillator screen to record the light image produced on the scintillator screen; providing a computer with a computer screen and software; capturing and analyzing the recorded light images from the camera; and finally obtaining a diagnosis for treatment of the patient.

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims attached.

In this respect, before explaining at least one embodiment of the invention in detail, it is to be understood that the invention is not limited in its application to the details of construction and to the arrangements of the components and steps set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an object of the present invention to provide a new and improved detection of COVID-19 method, which has all of the advantages of the prior art imaging methods of known designs and configurations and none of the disadvantages. It is another object of the present invention to provide a new and improved detection of COVID-19 system and method which may be easily and efficiently manufactured, marketed, and utilized. It is a further object of the present invention to provide a new and improved detection of COVID-19 method which is durable and reliable.

An even further object of the present invention is to provide a new and improved detection of COVID-19 method which is susceptible of a low cost of manufacture with regard to both materials and labor, and which accordingly is then susceptible of low prices of sale to medical professionals, thereby making such detection of COVID-19 method economically available to the public.

Lastly, it is an object of the present invention to provide a detection of COVID-19 method, for allowing medical professionals to easily visualize aspects presently hidden to them on current imaging systems.

These together with other objects of the invention, along with the various features of novelty which characterize the invention, are pointed out with particularity in the claims annexed to and forming a part of this disclosure.

For a better understanding of the invention, its operating advantages and the specific objects attained by its uses, reference should be had to the accompanying drawings and descriptive matter illustrating preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 shows a block diagram of components of the system for carrying out the method of the present invention.

FIG. 2 shows an arrangement of components in the detection of COVID-19 method of the present invention.

The same reference numerals refer to the same parts throughout the various Figures.

### DESCRIPTION OF THE PREFERRED EMBODIMENT

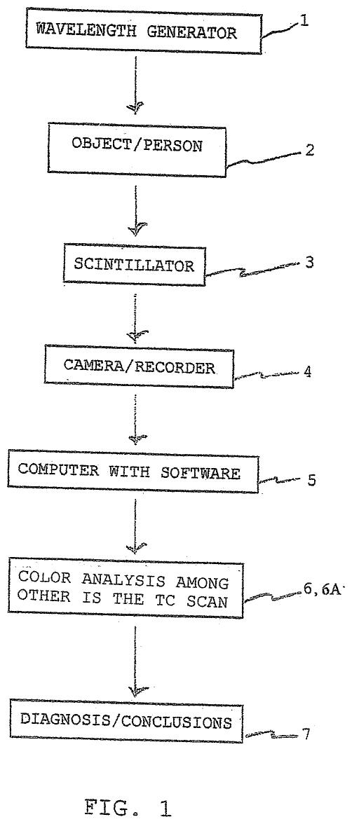

With reference now to the drawings, and in particular to FIG. 1 thereof, the preferred embodiment of the new and improved detection of COVID-19 method embodying the principles and concepts of the present invention and generally designated by the reference numeral 10 will be described.

The present invention, the detection of COVID-19 method is comprised of a plurality of steps. Such steps in their broadest context include providing a generator, providing a scintillator, positioning a patient, emitting a specific wavelength, providing a camera, providing a computer, drawing a conclusion and obtaining a diagnosis for treatment of a patient. Such components are individually configured and correlated with respect to each other so as to attain the desired objective. From a specific perspective, the invention of the present application is a medical diagnosis method comprising a plurality of steps. In the preferred embodiment, the first step is providing a generator adapted to produce specific wavelengths to maximize light emissions.

A scintillator is provided in operative proximity to the generator. The scintillator has an associated scintillator screen of a specific phosphor type sensitive to various wavelengths. The scintillator is sensitive to the specific wavelength that maximizes light emissions.

The next step is positioning a patient to be diagnosed between the generator and the scintillator.

The next step is emitting the specific wavelength from the generator to and through the patient onto the scintillator screen whereby the associated scintillator screen will light up and sparkle to produce a light image of the patient. The specific wavelength to be used is in the radio frequency, RF, range of the electromagnetic spectrum, EMS, to be captured and analyzed. The next step is providing a camera in operative proximity to the scintillator screen to record the light images produced on the scintillator screen.

The next step is providing a computer having a computer screen and software. The computer is adapted to capture and analyze the recorded light images from the camera. The software will analyze various light parameters from the image, such as hue and saturation corresponding to disease and inflammation revealing chronic, acute, and repairing items. This is an important and unique aspect of the present invention.

The final step is drawing a conclusion and obtaining a diagnosis for treating the patient.

The present invention is a method for using computer-based software and associated hardware system that allows medical professionals to easily visualize aspects presently hidden to them on current imaging systems. This method is extremely reasonable and simple to use. It will interface with existing imaging systems that do not allow such imaging and allows those systems to obtain information previously not accessible.

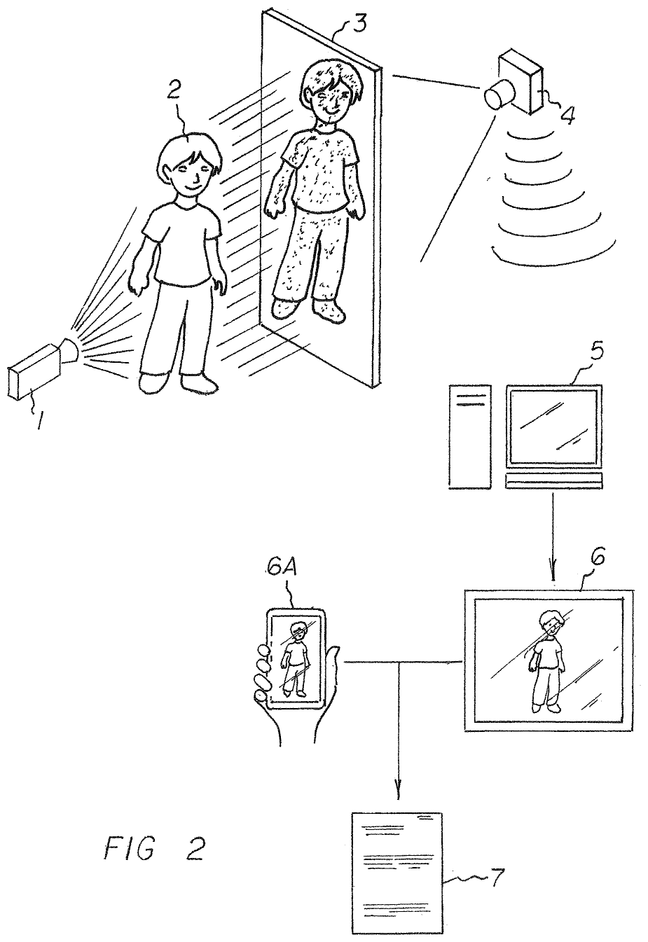

Furthermore, the system will be only a fraction of the cost of existing systems that allow medical professionals to check for disease in the body. Unlike these systems, the present invention does not require markers, dyes, or radioactive elements. Neither does it require injection of the former elements into the body. FIGS. 1 and 2 show an arrangement of the components of a configuration for carrying out the method of the present invention. The Figures show a generator 1 capable of producing specific wavelengths to maximize light emission onto a patient 2. Behind the patient is a scintillator 3. The scintillator has a scintillator screen of a specific phosphor or other excitive media type sensitive to various wavelengths. The scintillator screen is sensitive to a wavelength that maximizes light emission. The scintillator lights up and sparkles to produce a light image of the patient. A camera 4 records the light image presented on the scintillator screen. A computer 5 with the proper software captures, records, and analyzes the image from the camera. The software will analyze various light parameters from the image such as hue and saturation and display an output on a computer screen 6 or separate handheld device 6A with an associated printout 7 for an investigator to obtain a diagnosis and conclusion.

It uses a form of light, hereinafter referred to as HSTPi, selected from the light wave group including the Pi wave. The Pi wave is an extremely important part of the form of light. The wave group to be seen by the inspector is hereinafter referred to as HSTPiCOG. The device involved with the manipulation of the Pi wave is hereinafter referred to as the TCSCAN which, like other known imaging devices, utilizes the energy from the electromagnetic spectrum to aid mankind in investigations.

The present invention will help allay the world wide pandemic caused by COVID-19 virus. Its importance as a detector of the COVID-19 condition will allow for the world to set aside draconian methods such as the need to quarantine the populace rather than only those infected.

The world at this time, especially the United States of America needs a detection method that works when people are not showing signs of the disease. A testing method in the United States of America must be developed within a couple of weeks at most before we are in a disaster. Other countries such as Italy and Spain and the European Union are closing borders and some are placing their populace in quarantine in their homes.

The present invention detects the possibility of a COVID-19 viral infection during a state where the host does not show clinical signs. Present guidelines today say that there is no need to test without signs. Of course, this type of reasoning must change and probably is a result of not having testing methods set up for large masses of people to be tested. The present invention will also be helpful in looking at people with clinical cases of the disease to determine if healing is occurring. Information from clinically diseased people will be added to the detector's computer recorder so that the disease can be understood.

The invention is far superior to any type of detection in today's war with the virus. The detection methods today rely on testing conditions with many drawbacks, such as polymerase chain reaction (PCR) testing with high cost; low supplies; hours to gain results; false results; and objectionable invasive methods. The population may question or object to collection of buccal swabs in the current testing being used by the government because of possible DNA collection. Another detection method is use of temperature infrared (IR) thermometer where a febrile condition will cause an increase in the former, but occurs unfortunately after the host has passed through a period of infectivity to others without having a temperature. The thermometer fails to detect the unobvious condition of infected hosts. The last detection method proposed is with the CTt scan that is impractical and inferior to the present invention. It requires extremely expensive non-mobile machines that have to be analyzed by highly trained doctors or technicians. Its black and white image is a drawback and inferior to the color image that has relevance in the present invention. The present invention is capable of using color to detect the presence of a pathological agent leading to specific degrees of inflammation.

The invention is related to another patent application pending by the inventor, U.S. Patent Application No. 62/982,404 filed Feb. 27, 2020. That invention uses the Pi wave, but that wave and the wave group HSTPi is hyperdriven through newly discovered means. The HSTPi will be used in the medical and legal field to answer questions of inflammatory and immunological disturbances. The wave group to be seen by the inspector, hereinafter HSTPi, with the Pi component is of great importance.

This invention for screening has elements the other patent application does not and has been designed to be the best type of detector especially with safety requirement. This device is for detection the other (application) is for medical diagnosis, healing and therapeutics. The invention described here is not inferior to anything known to man. It has been adapted with to give a superior method to detect. It offers great safety in its use compared to other imaging modalities; daily tests possible to those given in a working environment such as government buildings or airports or schools on a daily basis; great accuracy; extremely low cost; ease of mobility; and immediate results with no need to have specialized training.

The present invention will allow the populace to accomplish the mission of detecting at a level that will suffice, but is not so developed as that used in the medical and legal field to answer questions of inflammatory and immunological disturbances. The wave group to be seen by the inspector named HSTPi with the Pi component of great importance.

The present invention allows health departments and independent clinics working together with the use of software, hardware, and a somewhat modern digital x-ray to develop a working TCSCAN. The required equipment can be put together for little cost and in a rapid manner. It is extremely important because it allows the United States of America and the world to rapidly but together a sufficient TCSCAN in every country, state, province, city and town with no great expense and in a few short hours—at most. The present invention will satisfy the need to detect and free up our populace that are clear of the virus. It is thinking globally but acting locally.

Following are some of the reasons that the present invention is extremely important and urgently needed:

1) It detects the presence of an inflammatory agent that has been linked to people carrying the virus. It is a detector that can be used in the earliest stage of infection where the host is capable of spreading the disease without clinical signs.

2) If the inflammatory agent in the chest is not identified, that person will continue working in society and not be confined for weeks at home or an institution or camp. The confinement points just mentioned may actually increase the chances for a person without the disease to actively contract the disease from others that may be positive in the confinement areas.

3) It will allow the medical community to begin the first and vital part of the epidemiological study which is the ability to detect the agent of disease. At this time, modern medicine is using methods of the dark ages and earlier by confining any and all suspected or showing signs to facilities like it was done in leper colonies or to self isolation. For all people to be placed among others not tested but in isolation is not logical and frankly malpractice.

4) The United States at this moment falls behind most other countries in the testing, numbers of tests, and detection abilities.

5) Detecting people who are normal without the virus and allowing such uninfected people to continue to work reduces the chances of us being driven into an economic collapse and/or a full blown pandemic.

Aspects of the current invention are presented below in progressive steps.

1) Use of a wavelength generator
unto an object or person
the wave passes through the object
goes to a scintillator
the light is enhanced and emitted at predetermined wavelengths
wave group is selected named HSTPiCOG
light goes to a camera or recorder with an augmented LED to pixel ratio (made possible by the use of previously unknown steric changes of the LED) and/or number of LEDs
augmentation of the pixel-LED ratio set to an optimum position or setting
light is maximized or hyperdriven
light produced is further adjusted through lasers
direction maximized (further light enhancement)
goes to a computer or recorder
gathered with high quality detectors
software alterations of hue/saturation separates the light into bands of light
Bands expressed as extremely small numerical values to further represent extremely fine differences in the intensities of light
software converts numerical values to color and/or
sends numbers to be evaluated and compared to other numbers associated with the disease and disease agents
numerical values sent to be viewed as color are made to be within predetermined gradients with a minimal overlap or bleed between the colors. For example, the color “red” rather than being seen as red will have many forms of red that will represent factors of the HSTPiCOG wave form
final result is digitized or left as a color wave-form
inflammation and agent presence is detected by the investigator
audible sounds may be made and other digital transmission may be sent to authorities if a positive color for infection is detected
transferring information over to a digital bank to use in epidemiological studies if deemed necessary.

The instructions showing the steps and components used for the detection of COVID-19 using the present invention are:

a digital x-ray or c-arm of high quality
attach a separate computer to it
attach a high end LED monitor, the high end monitor being selected from the group of high end monitors including Samsung IT C27F398, Dell UltraSharp U2414H, ViewSonic VX2452MH, Acer G276HL Kbix, ASUS VS248H-P; LG 34 UC80-B, LG 32MA70HY-P, AC34 SFA240k HP FHD IPS, and HP Pavilion IPS.
download a computer software program for hue/saturation
send the picture from the x-ray in JPEG format to the attached computer
open the computer software program for hue/saturation
right click the JPEG picture and open it on your high end LED monitor in the computer software program for hue/saturation
hit image and then image size, the preferred image size being 4 or 5 inches height
proceed while still under image and go up in image and hit Adjustments then find hue/saturation
move the hue to the right to maximum 180, the saturation to 100, lightness
hit custom and save this preset as TCSCAN. Note that other colors can be developed but these readings are for the colors that have demonstrated to refer to the actual state of inflammation on reverse studies.
Reopen and you will see default, go down on right and find TCSCAN and you should now see the waveform. Pressing the JPEG on your opened program you will be asked questions such as optimum or basic, hit optimum.
picture will be sent to your desktop in the attached monitor.

The use of a proper monitor is essential for the detection of COVID-19 of the present invention. The monitor must have a good LED/pixel ratio in order to colorize and continue giving the color change of the hyper-driven light. The invention is considered relatively safe at these low levels that are later intensified by the image intensifier on the machine. A lower kV can be used to machines properly adapted to reduce any dangers that the public may associate with chest x-rays. It is not necessary to see every branch of the lung, just a color over the chest, red or yellow or a little brown. Reducing the x-ray is OK in the present invention. Use of a c-arm is acceptable with lower kV.

As to the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed as being new and desired to be protected by Letters Patent of the United States is as follows:

1. A medical diagnosis method comprising the steps of:
providing a generator adapted to produce specific wavelengths to maximize light emissions;
providing a scintillator in operative proximity to the generator, the scintillator having an associated scintillator screen of an excitive media type including a specific phosphor type sensitive to various wavelengths, the scintillator being sensitive to a wavelength that maximizes light emissions;
positioning a patient to be diagnosed between the generator and the scintillator;
emitting a specific wavelength from the generator to and through the patient onto the scintillator screen whereby the associated scintillator screen will light up and sparkle to produce a light image of the patient or reflect back from the back boundary of the patient to a scintillator in the receiver;
providing a camera in operative proximity to the scintillator screen to record the light image produced on the scintillator screen;
providing a computer with an aftermarket computer screen provided with greatly unheard number of LED's and a software designed and capable of capturing and analyzing the light intensity associated with the LED numbers, the recorded light images from the camera will allow a person to draw a repeatable conclusion from the degree of color intensity associated with the Pi wave and inflammation; and
drawing a conclusion and obtaining a diagnosis which is repeatable and sustainable and allows for diagnosis and treatment of the patient.

2. The method as set forth in claim 1 wherein the specific wavelength is in the radio frequency range of the electromagnetic spectrum to thereby reveal color and intensity corresponding to disease and inflammation to discern chronic, acute, and repairing items.

3. The method as set forth in claim 1 wherein the computer screen is an LED monitor.

4. The method as set forth in claim 1 wherein the computer screen is a separate handheld device.

5. A medical diagnosis method comprising the steps of:
providing a generator adapted to produce a specific wavelength to maximize light emissions;
providing a scintillator in operative proximity to the generator, the scintillator having an associated scintillator screen of a specific phosphor type sensitive to various wavelengths, the scintillator being sensitive to the specific wavelength;
positioning a patient to be diagnosed between the generator and the scintillator;
emitting the specific wavelength from the generator to and through the patient onto the scintillator screen whereby the associated scintillator screen will light up and sparkle to produce a light image of the patient between the generator and the scintillator or reflected from the patient onto the scintillator, the EMS wavelength to be used is in EMS associated with various frequencies deemed useful for the particular investigator such as RF, x-ray UV ranges of the electromagnetic spectrum, to be captured and analyzed;
providing a camera in operative proximity to the scintillator screen to record the light images produced on the scintillator screen; and
providing a computer having a computer screen and software, the computer adapted to capture and analyze the recorded light images from the camera, the software adapted to analyze various precise and fine light parameters from the image that will reveal hue and saturation corresponding to disease and inflammation, chronic, acute, and repairing elements; and
drawing a conclusion and obtaining a diagnosis for use in treating the patient.

6. A detection of COVID-19 method comprising the steps of:
providing a wavelength generator;
emitting the wavelength unto and through an object or person;
providing a scintillator;
providing a light from a wave group, the wave group including a Pi wave and referred to as HSTPiCOG;
enhancing the light at predetermined wavelengths;
sending the light to a camera or recorder with an augmented LED to pixel ratio and/or number of LEDs;
augmenting the pixel-LED ratio set to an optimum position or setting;
maximizing and hyperdriving the light;
adjusting the light produced through lasers;
maximizing direction and further enhancing the light;
sending the light to a computer or recorder having an LED monitor;
gathering the light with high quality detectors;
separating the light into bands of light using software alterations of hue/saturation;
expressing the bands as numerical values to further represent extremely fine differences in the intensities of the light;
converting numerical values to color using the software;
sending numbers to be evaluated and compared to other numbers associated with the COVID-19 virus and disease agents;
sending numerical values to be viewed as color within predetermined gradients with a minimal overlap or bleed between the colors for a color wave-form, hereinafter referred to as an HSTPiCOG wave-form;
digitizing the final result;
detecting inflammation and agent presence;
producing audible sounds and other digital transmission to authorities if a positive color for infection is detected; and
transferring information over to a digital bank to use in epidemiological studies.
